(19) United States
(12) Patent Application Publication
(10) Pub. No.: US 2024/0094475 A1
(43) Pub. Date: Mar. 21, 2024

(54) Disinfecting Covers for Functional Connectors of Medical Devices and Methods Thereof

(71) Applicant: Bard Access Systems, Inc., Salt Lake City, UT (US)

(72) Inventors: Anthony K. Misener, Bountiful, UT (US); Steffan Sowards, Salt Lake City, UT (US)

(21) Appl. No.: 18/524,620

(22) Filed: Nov. 30, 2023

Related U.S. Application Data
(63) Continuation of application No. 17/499,644, filed on Oct. 12, 2021.
(60) Provisional application No. 63/091,165, filed on Oct. 13, 2020.

(51) Int. Cl. G02B 6/38

(52) U.S. Cl. CPC G02B 6/3866; G02B 6/3849; A61B 46/10

(57) ABSTRACT

Disclosed are disinfecting covers for optical-fiber connectors. For example, a male disinfecting cover can include a plug, a bore of the plug, an absorbent disposed in the bore, and a disinfectant absorbed by the absorbent. The plug is configured to insert into a receptacle of a female optical-fiber connector. A female disinfecting cover can include a body, a receptacle in the body, an absorbent disposed in the receptacle, and a disinfectant absorbed by the absorbent. The receptacle is configured to accept a male optical-fiber connector. Whether the disinfecting cover is male or female, the absorbent is configured to contact the ferrule and the optical fiber disposed of the optical-fiber connector. Methods can include at least a method of using the male or female disinfecting cover.

### PRIORITY

This application is a division of U.S. patent application Ser. No. 17/499,644, filed Oct. 12, 2021, which claims the benefit of priority to U.S. Provisional Application No. 63/091,165, filed Oct. 13, 2020, each of which is incorporated by reference in its entirety into this application.

### BACKGROUND

Some multiple-use medical devices are used in relatively close proximity to patients so that the multiple-use medical devices can be functionally connected to single-use medical devices by functional connectors thereof. Being in close proximity to the patients, there are risks of contaminating the multiple-use medical devices with, for example, bodily fluids or bodily fluid-borne pathogens, as well as spreading such contamination to subsequent patients or other clinical areas in which the multiple-use medical devices are used. What is needed are disinfecting covers for functional connectors of the multiple-use medical devices that disinfect the functional connectors between uses.

Disclosed herein are disinfecting covers for functional connectors of medical devices and methods thereof that address the foregoing.

### SUMMARY

Disclosed herein is a disinfecting cover for an optical-fiber connector including, in some embodiments, a plug, a bore of the plug, an absorbent disposed in the bore, and a disinfectant absorbed by the absorbent. The plug is configured to insert into a receptacle of the optical-fiber connector. The absorbent is configured to contact a ferrule of the optical-fiber connector. The ferrule has an optical fiber disposed therein. The disinfectant is configured to disinfect at least the ferrule and the optical fiber.

In some embodiments, the disinfecting cover further includes one or more interlocking features. The one-or-more interlocking features are configured to interlock with the optical-fiber connector and maintain the plug in the receptacle of the optical-fiber connector when the disinfecting cover is inserted in the optical-fiber connector.

In some embodiments, the disinfecting cover further includes a handle opposite the plug. The handle is configured to enable a person to insert the plug into the receptacle of the optical-fiber connector or remove the plug from the receptacle of the optical-fiber connector by way of the handle.

In some embodiments, the plug and handle are integrally molded from a thermoplastic. The thermoplastic is selected from acrylonitrile butadiene styrene, polyethylene, polycarbonate, polyamide, high-impact polystyrene, and polypropylene.

In some embodiments, the absorbent is a compressible sponge of polyester, polyurethane, or cellulose.

In some embodiments, the sponge is configured to release a portion of the disinfectant absorbed thereby when compressed into the bore of the plug by the ferrule of the optical-fiber connector.

In some embodiments, the disinfectant is an aqueous solution of isopropanol.

In some embodiments, the solution is at least 70% isopropanol by volume.

In some embodiments, the disinfecting cover further includes a communication module. The communication module is configured to communicate with a medical device including the optical-fiber connector and indicate to the medical device when the optical-fiber connector is covered by the disinfecting cover.

In some embodiments, a cleaning cycle including cleaning or drying is initiated upon the communication module indicating to the medical device the optical-fiber connector is covered by the disinfecting cover.

In some embodiments, actuation of a cleaning action is initiated upon the communication module indicating to the medical device the optical-fiber connector is covered by the disinfecting cover.

In some embodiments, the disinfecting cover is integrated into a portal in a procedural barrier for a medical procedure. The portal is configured to enable functional connections between medical devices on opposing sides of the procedural barrier.

Also disclosed herein is a disinfecting cover for an optical-fiber connector including, in some embodiments, a body, a receptacle in the body, an absorbent disposed in the receptacle, and a disinfectant absorbed by the absorbent. The receptacle is configured to accept the optical-fiber connector therein. The absorbent is configured to contact a ferrule of the optical-fiber connector. The ferrule has an optical fiber disposed therein. The disinfectant is configured to disinfect at least the ferrule and the optical fiber.

In some embodiments, the disinfecting cover further includes one or more interlocking features. The one-or-more interlocking features are configured to interlock with the optical-fiber connector and maintain the optical-fiber connector in the receptacle of the body when the optical-fiber connector is inserted in the disinfecting cover.

In some embodiments, the disinfecting cover further includes a handle incorporated into the body or extending therefrom. The handle is configured to enable a person to insert the optical-fiber connector into the receptacle of the body or remove the optical-fiber connector from the receptacle when holding the disinfecting cover by the handle.

In some embodiments, the body and handle are integrally molded from a thermoplastic. The thermoplastic is selected from acrylonitrile butadiene styrene, polyethylene, polycarbonate, polyimide, high-impact polystyrene, and polypropylene.

In some embodiments, the absorbent is a compressible sponge of polyester, polyurethane, or cellulose.

In some embodiments, the sponge is configured to release a portion of the disinfectant absorbed thereby when compressed into the receptacle of the body by the ferrule of the optical-fiber connector.

In some embodiments, the disinfectant is an aqueous solution of isopropanol.

In some embodiments, the solution is at least 70% isopropanol by volume.

In some embodiments, the disinfecting cover further includes a communication module. The communication module is configured to communicate with a medical device including the optical-fiber connector and indicate to the medical device when the optical-fiber connector is covered by the disinfecting cover.

In some embodiments, a cleaning cycle including cleaning or drying is initiated upon the communication module indicating to the medical device the optical-fiber connector is covered by the disinfecting cover.

In some embodiments, actuation of a cleaning action is initiated upon the communication module indicating to the medical device the optical-fiber connector is covered by the disinfecting cover.

In some embodiments, the disinfecting cover is integrated into a portal in a procedural barrier for a medical procedure. The portal is configured to enable functional connections between medical devices on opposing sides of the procedural barrier.

Also disclosed herein is a method for disinfecting optical-fiber connectors. The method includes, in some embodiments, a disconnecting step, a first inserting step, a second inserting step, and a storing step. The disconnecting step includes disconnecting a male optical-fiber connector from a female optical-fiber connector. The first inserting step includes inserting the male optical-fiber connector into a female disinfecting cover, thereby disinfecting at least a ferrule and an optical fiber of the male optical-fiber connector. The second inserting step includes inserting a male disinfecting cover into a female optical-fiber connector, thereby disinfecting at least a ferrule and an optical fiber of the female optical-fiber connector. The storing step includes keeping the male optical-fiber connector or the female optical-fiber connector in its respective disinfecting cover until connecting the male optical-fiber connector or the female optical-fiber connector to each other or another complementary optical-fiber connector.

In some embodiments, the first inserting step includes inserting the male optical-fiber connector into a receptacle of a body of the female disinfecting cover. The receptacle includes an absorbent having a disinfectant absorbed by the absorbent.

In some embodiments, the first inserting step includes compressing the absorbent into the receptacle, thereby releasing a portion of the disinfectant for the disinfecting of the male optical-fiber connector.

In some embodiments, the second inserting step includes inserting a plug of the disinfecting cover into a receptacle of the female optical-fiber connector. The plug includes a bore with an absorbent having a disinfectant absorbed by the absorbent.

In some embodiments, the second inserting step includes compressing the absorbent into the bore, thereby releasing a portion of the disinfectant for the disinfecting of the female optical-fiber connector.

In some embodiments, the method mitigates contamination of multiple-use medical devices between uses with different patients.

These and other features of the concepts provided herein will become more apparent to those of skill in the art in view of the accompanying drawings and following description, which describe particular embodiments of such concepts in greater detail.

### BRIEF DESCRIPTION OF THE DRAWINGS

FIG. 1 illustrates functionally connected single-use and multiple-use medical devices of an optical shape-sensing system in accordance with some embodiments.

FIG. 2 illustrates functional connectors of the multiple-use medical devices of FIG. 1 in accordance with some embodiments.

FIG. 3 illustrates a female disinfecting cover for a male optical-fiber connector in accordance with some embodiments.

FIG. 4 illustrates a male disinfecting cover for a female optical-fiber connector in accordance with some embodiments.

FIG. 5 illustrates inserting a male optical-fiber connector into the female disinfecting cover in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

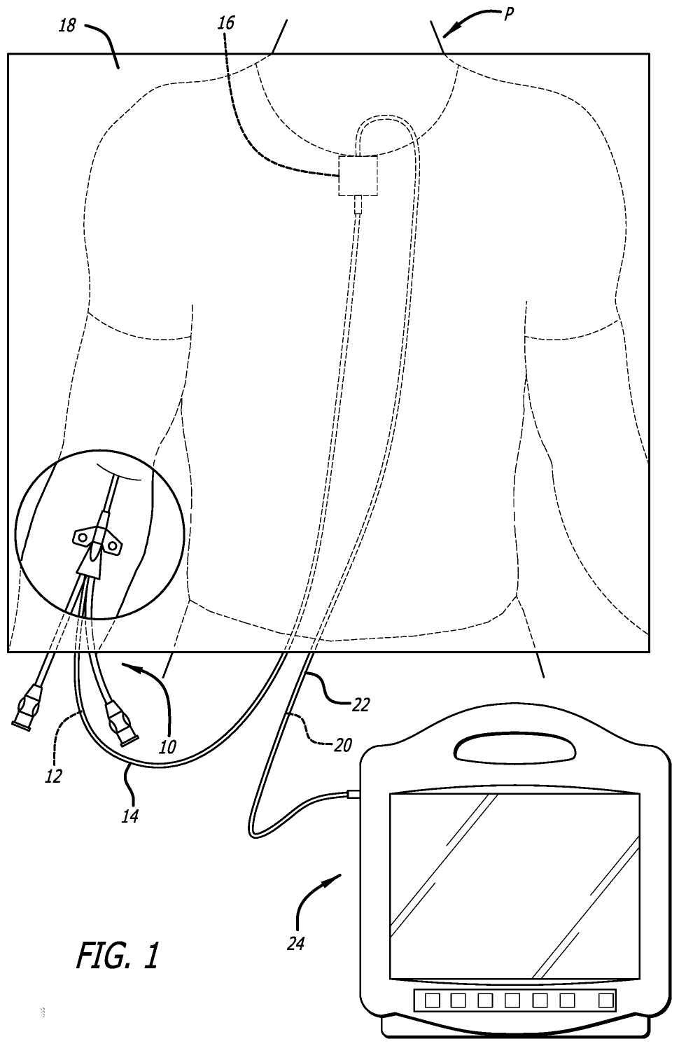

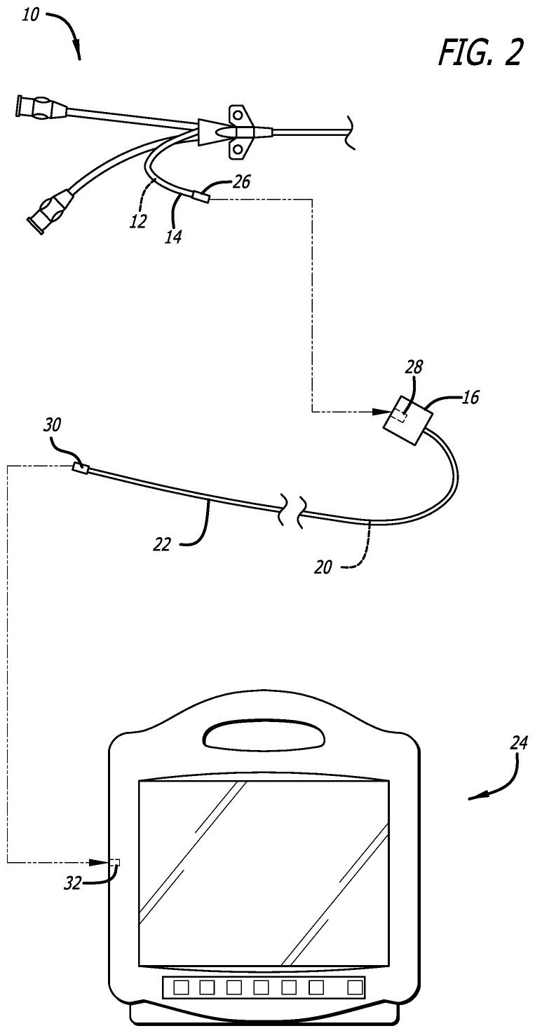

As set forth above, some multiple-use medical devices are used in relatively close proximity to patients so that the multiple-use medical devices can be functionally connected to single-use medical devices by functional connectors thereof. For example, FIG. 1 illustrates an optical shape-sensing system including a single-use peripherally inserted central catheter (“PICC”) 10 having an optical-fiber stylet 12 disposed in an extension tube 14 functionally connected to a relay module 16 over a patient P but under a sterile drape 18. The relay module 16, in turn, includes an optical fiber 20 disposed in a patch cable 22 functionally connected to a console 24 or an optical interrogator thereof. FIG. 2 illustrates functional connectors of the foregoing single-use and multiple-use medical devices including a male optical-fiber connector 26 of the PICC 10, a female optical-fiber connector 28 of the relay module 16, a male optical-fiber connector 30 of the patch cable 22, and the female optical-fiber connector 32 of the console 24 or the optical interrogator thereof. With the multiple-use medical devices such as the relay module 16, the patch cable 22, and the console 24 being in close proximity to the patients, there are risks of contaminating the multiple-use medical devices with, for example, bodily fluids or bodily fluid-borne pathogens, as well as spreading such contamination to subsequent patients or other clinical areas in which the multiple-use medical devices are used. Again, disinfecting covers for functional connectors of the multiple-use medical devices that disinfect the functional connectors between uses are needed.

Disclosed herein are disinfecting covers for functional connectors of medical devices and methods thereof. While the disinfecting covers and the methods thereof are primarily described for optical shape-sensing systems, the disinfecting covers and the methods thereof are not limited thereto. Indeed, some medical devices include electrical connectors instead of optical connectors or in addition to optical connectors. Whether the functional connectors are optical connectors, electrical connectors, or a combination thereof, the need for the disinfecting covers for the functional connectors and methods thereof remains. Thus, it should be understood the concepts provided herein for optical-fiber connectors can be extended to electrical connectors or combinations thereof to the same effect as set forth herein for the optical-fiber connectors.

### Disinfecting Covers

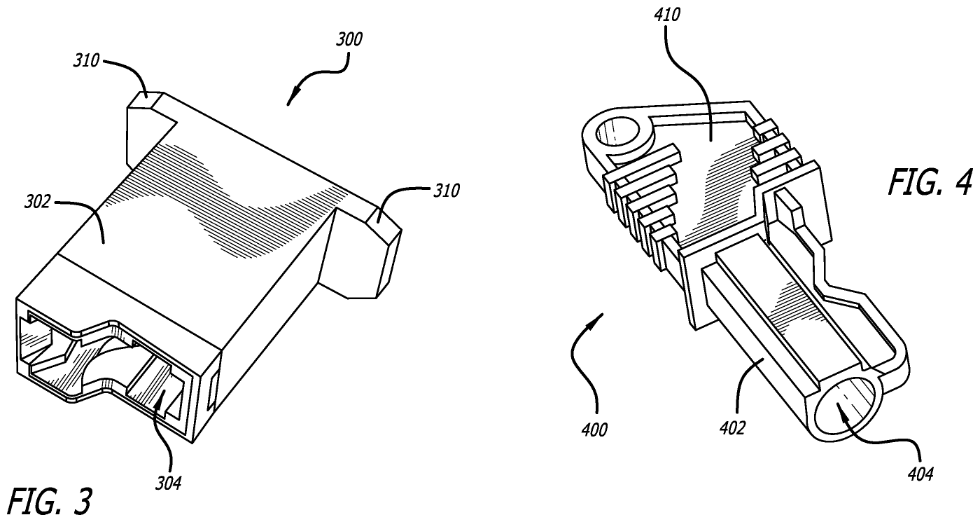

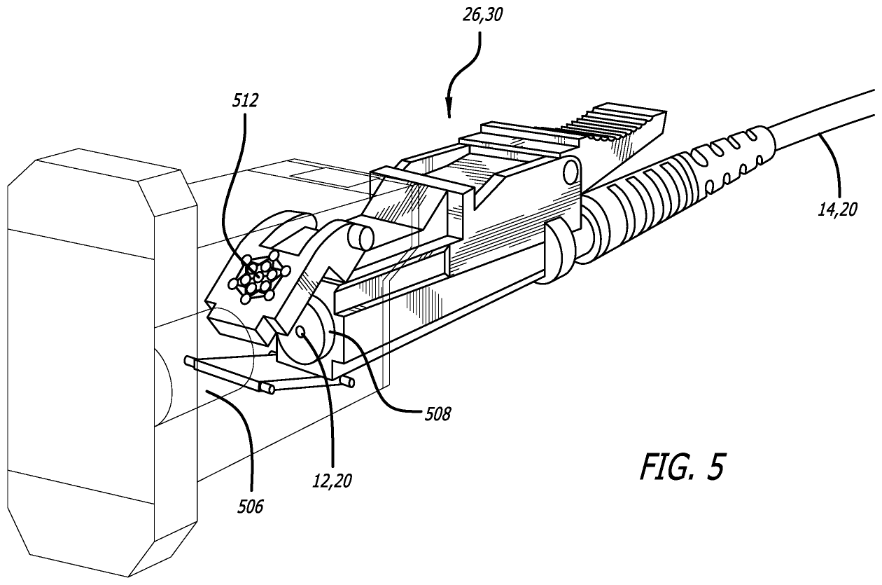

FIG. 3 illustrates a female disinfecting cover 300 for a male optical-fiber connector in accordance with some embodiments. FIG. 5 illustrates inserting a male optical-fiber connector into the female disinfecting cover 300 in accordance with some embodiments.

As shown, the female disinfecting cover 300 is configured to accept a male optical-fiber connector such as the male optical-fiber connector 26 or 30 inserted therein for disinfecting the male optical-fiber connector 26 or 30, as well as protecting the male optical-fiber connector 26 or 30 from dust.

The female disinfecting cover 300 includes a body 302, a receptacle 304 in the body 302, an absorbent 506 disposed in the receptacle 304, and a disinfectant absorbed by the absorbent 506. As shown, the female disinfecting cover 300 can further include a handle 310, as well as one or more interlocking features generally shown. While there are many different male optical-fiber connectors, the male optical-fiber connector 26 or 30 is representative in that it includes a ferrule 508 having the optical fiber 12 or 20 disposed therein. The absorbent 560 is configured to contact the ferrule 508 and the optical fiber 12 or 20 disposed therein when the male optical-fiber connector 26 or 30 is inserted into the female disinfecting cover 300. The disinfectant is configured to disinfect at least the ferrule 508 and the optical fiber 12 or 20; however, the female disinfecting cover 300 can be larger to accommodate more of the male optical-fiber connector 26 or 30 for disinfection thereof.

The handle 310 can be incorporated into the body 302 or extend therefrom. The handle 310 is configured to enable a person to insert the male optical-fiber connector 26 or 30 into the receptacle 304 or remove the male optical-fiber connector 26 or 30 from the receptacle 304 when holding the female disinfecting cover 300 by the handle 310.

The one-or-more interlocking features (e.g., locking channels, clips, threads, etc.) are configured to interlock with the male optical-fiber connector 26 or 30 and maintain the male optical-fiber connector 26 or 30 in the receptacle 304 when the male optical-fiber connector 26 or 30 is inserted in the female disinfecting cover 300. In addition, with respect to at least E-2000-type male optical-fiber connectors such as the optical-fiber connector 26 or 30, the one-or more interlocking features can be further configured to expose the ferrule 508 and the optical fiber 12 or 20 by moving a cover 512 thereof away from the ferrule 508 and the optical fiber 12 or 20. Again, there are many different male optical-fiber connectors, so the one-or-more interlocking features can vary accordingly.

The body 302, the handle 310, and the one-or-more interlocking features can be integrally molded from a thermoplastic or separately molded and snapped or bonded together. The thermoplastic is selected from acrylonitrile butadiene styrene, polyethylene, polycarbonate, polyimide, high-impact polystyrene, and polypropylene.

The absorbent 506 can be a compressible sponge of polyester, polyurethane, or cellulose. Whether or not the absorbent 506 is a sponge, the absorbent 506 is configured to release a portion of the disinfectant absorbed thereby when compressed into the receptacle 304 by the ferrule 508 of the male optical-fiber connector 26 or 30.

The disinfectant can be an aqueous solution of isopropanol or an iodophor. When the disinfectant is the solution of isopropanol, the solution is at least 70% isopropanol by volume.

The female disinfecting cover 300 can further include a communication module. The communication module is configured to communicate with a medical device including the male optical-fiber connector and indicate to the medical device when the male optical-fiber connector is covered by the female disinfecting cover 300. A cleaning cycle including cleaning or drying is initiated upon the communication module indicating to the medical device the male optical-fiber connector is covered by the female disinfecting cover 300. Actuation of a cleaning action is initiated upon the communication module indicating to the medical device the male optical-fiber connector is covered by the female disinfecting cover 300.

The female disinfecting cover 300 can be integrated into a portal in a procedural barrier for a medical procedure. The portal is configured to enable functional connections between medical devices on opposing sides of the procedural barrier.

FIG. 4 illustrates a male disinfecting cover 400 for a female optical-fiber connector in accordance with some embodiments.

As shown, the male disinfecting cover 400 is configured to insert into a receptacle of a female optical-fiber connector such as the female optical-fiber connector 28 or 32 for disinfecting the female optical-fiber connector 28 or 32, as well as protecting the female optical-fiber connector 28 or 32 from dust.

The male disinfecting cover 400 includes a plug 402, a bore 404 of the plug 402, an absorbent disposed in the bore 404, and a disinfectant absorbed by the absorbent. While the absorbent is not shown in FIG. 4, the absorbent is analogous to the absorbent 506 of the female disinfecting cover 300. As shown, the male disinfecting cover 400 can further include a handle 410 opposite the plug 402, as well as one or more interlocking features generally shown. While there are many different female optical-fiber connectors, the female optical-fiber connector 28 or 32 is representative in that it includes a ferrule having an optical fiber disposed therein. The absorbent is configured to contact the ferrule and the optical fiber disposed therein when the male disinfecting cover 400 is inserted into the receptacle of the female optical-fiber connector 28 or 32. Indeed, the bore 404 is configured to accept therein the ferrule of the female optical-fiber connector 28 or 32 to establish such contact. The disinfectant is configured to disinfect at least the ferrule and the optical fiber.

The handle 410 is configured to enable a person to insert the plug 402 into the receptacle of the female optical-fiber connector 28 or 32 or remove the plug 402 from the receptacle of the female optical-fiber connector 28 or 32 by way of the handle 410 when holding the male disinfecting cover 400 by the handle 410.

The one-or-more interlocking features (e.g., locking channels, clips, threads, etc.) are configured to interlock with the female optical-fiber connector 28 or 32 and maintain the plug 402 in the receptacle of the female optical-fiber connector 28 or 32 when the plug 402 of the male disinfecting cover 400 is inserted in the female optical-fiber connector 28 or 32. Again, there are many different female optical-fiber connectors, so the one-or-more interlocking features can vary accordingly.

The plug 402, the handle 410, and the one-or-more interlocking features can be integrally molded from a thermoplastic or separately molded and snapped or bonded together. The thermoplastic is selected from acrylonitrile butadiene styrene, polyethylene, polycarbonate, polyamide, high-impact polystyrene, and polypropylene.

As set forth above, the absorbent can be a compressible sponge of polyester, polyurethane, or cellulose. Whether or not the absorbent is a sponge, the absorbent is configured to release a portion of the disinfectant absorbed thereby when compressed into the bore 404 by the ferrule of the female optical-fiber connector 28 or 32.

The disinfectant can be an aqueous solution of isopropanol or an iodophor. When the disinfectant is the solution of isopropanol, the solution is at least 70% isopropanol by volume.

The male disinfecting cover 400 can further include a communication module. The communication module is configured to communicate with a medical device including the female optical-fiber connector and indicate to the medical device when the female optical-fiber connector is covered by the male disinfecting cover 400. A cleaning cycle including cleaning or drying is initiated upon the communication module indicating to the medical device the female optical-fiber connector is covered by the male disinfecting cover 400. Actuation of a cleaning action is initiated upon the communication module indicating to the medical device the female optical-fiber connector is covered by the male disinfecting cover 400.

The male disinfecting cover 400 can be integrated into a portal in a procedural barrier for a medical procedure. The portal is configured to enable functional connections between medical devices on opposing sides of the procedural barrier.

### Methods

Methods include a method for disinfecting optical-fiber connectors such as the male optical-fiber connector 26 or 30 or the female optical-fiber connector 28 or 32. Such a method can include a disconnecting step, a first inserting step, a second inserting step, and a storing step.

The disconnecting step includes disconnecting a male optical-fiber connector from a female optical-fiber connector such as the male optical-fiber connector 26 from the female optical-fiber connector 28 or the male optical-fiber connector 30 from the female optical-fiber connector 32.

The first inserting step includes inserting the male optical-fiber connector 26 or 30 into the female disinfecting cover 300, thereby disinfecting at least the ferrule 508 and the optical fiber 12 or 20 of the male optical-fiber connector 26 or 30. Indeed, the first inserting step includes inserting the male optical-fiber connector 26 or 30 into the receptacle 304 of the female disinfecting cover 300. As set forth above, the receptacle 304 includes the absorbent 506 having the disinfectant absorbed by the absorbent 506. Thus, the first inserting step also includes compressing the absorbent 506 into the receptacle 304, thereby releasing a portion of the disinfectant for the disinfecting of the male optical-fiber connector 26 or 30.

The second inserting step includes inserting the male disinfecting cover 400 into the female optical-fiber connector 28 or 32, thereby disinfecting at least the ferrule and the optical fiber of the female optical-fiber connector 28 or 32. Indeed, the second inserting step includes inserting the plug 402 of the male disinfecting cover 400 into the receptacle of the female optical-fiber connector 28 or 32. As set forth above, the plug 402 includes the bore 404 with the absorbent having the disinfectant absorbed by the absorbent. Thus, the second inserting step includes compressing the absorbent into the bore 404, thereby releasing a portion of the disinfectant for the disinfecting of the female optical-fiber connector 28 or 32.

The storing step includes keeping the male optical-fiber connector 26 or 30 or the female optical-fiber connector 28 or 32 in its respective disinfecting cover until connecting the male optical-fiber connector 26 or 30 or the female optical-fiber connector 28 or 32 to each other or another complementary optical-fiber connector.

As set forth herein, the method mitigates contamination of multiple-use medical devices between uses with different patients.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A disinfecting cover for an optical-fiber connector, comprising:
a body;
a receptacle in the body configured to accept the optical-fiber connector therein;
an absorbent disposed in the receptacle configured to contact a ferrule of the optical-fiber connector having an optical fiber disposed therein; and
a disinfectant absorbed by the absorbent configured to disinfect at least the ferrule and the optical fiber.

2. The disinfecting cover of claim 1, further comprising one or more interlocking features configured to interlock with the optical-fiber connector and maintain the optical-fiber connector in the receptacle of the body when the optical-fiber connector is inserted in the disinfecting cover.

3. The disinfecting cover of claim 1, further comprising a handle incorporated into the body or extending therefrom, the handle configured to enable a person to insert the optical-fiber connector into the receptacle of the body or remove the optical-fiber connector from the receptacle when holding the disinfecting cover by the handle.

4. The disinfecting cover of claim 3, wherein the body and handle are integrally molded from a thermoplastic selected from acrylonitrile butadiene styrene, polyethylene, polycarbonate, polyamide, high-impact polystyrene, and polypropylene.

5. The disinfecting cover of claim 1, wherein the absorbent is a compressible sponge of polyester, polyurethane, or cellulose.

6. The disinfecting cover of claim 5, wherein the sponge is configured to release a portion of the disinfectant absorbed thereby when compressed into the receptacle of the body by the ferrule of the optical-fiber connector.

7. The disinfecting cover of claim 1, wherein the disinfectant is an aqueous solution of isopropanol.

8. The disinfecting cover of claim 7, wherein the solution is at least 70% isopropanol by volume.

9. The disinfecting cover of claim 1, further comprising a communication module configured to communicate with a medical device including the optical-fiber connector and indicate to the medical device when the optical-fiber connector is covered by the disinfecting cover.

10. The disinfecting cover of claim 9, wherein a cleaning cycle including cleaning or drying is initiated upon the communication module indicating to the medical device the optical-fiber connector is covered by the disinfecting cover.

11. The disinfecting cover of claim 9, wherein actuation of a cleaning action is initiated upon the communication module indicating to the medical device the optical-fiber connector is covered by the disinfecting cover.

12. The disinfecting cover of claim 1, wherein the disinfecting cover is integrated into a portal in a procedural barrier for a medical procedure, the portal configured to enable functional connections between medical devices on opposing sides of the procedural barrier.

13. A method for disinfecting optical-fiber connectors, comprising:
disconnecting a male optical-fiber connector from a female optical-fiber connector;
inserting the male optical-fiber connector into a female disinfecting cover, thereby disinfecting at least a ferrule and an optical fiber of the male optical-fiber connector;
inserting a male disinfecting cover into a female optical-fiber connector, thereby disinfecting at least a ferrule and an optical fiber of the female optical-fiber connector; and
keeping the male optical-fiber connector or the female optical-fiber connector in its respective disinfecting cover until connecting the male optical-fiber connector or the female optical-fiber connector to each other or another complementary optical-fiber connector.

14. The method of claim 13, wherein inserting the male optical-fiber connector into the female disinfecting cover includes inserting the male optical-fiber connector into a receptacle of a body of the female disinfecting cover, the receptacle including an absorbent having a disinfectant absorbed by the absorbent.

15. The method of claim 14, wherein inserting the male optical-fiber connector into the female disinfecting cover includes compressing the absorbent into the receptacle, thereby releasing a portion of the disinfectant for the disinfecting of the male optical-fiber connector.

16. The method of claim 13, wherein inserting the male disinfecting cover into the female optical-fiber connector includes inserting a plug of the disinfecting cover into a receptacle of the female optical-fiber connector, the plug including a bore with an absorbent having a disinfectant absorbed by the absorbent.

17. The method of claim 16, wherein inserting the male disinfecting cover into the female optical-fiber connector includes compressing the absorbent into the bore, thereby releasing a portion of the disinfectant for the disinfecting of the female optical-fiber connector.

18. The method of claim 13, wherein the method mitigates contamination of multiple-use medical devices between uses with different patients.
